(19) United States
(12) Patent Application Publication
(10) Pub. No.: US 2024/0352391 A1
(43) Pub. Date: Oct. 24, 2024

(54) FLUID FLOW PLATE

(71) Applicant: Newcells Biotech Limited, Newcastle upon Tyne, Tyne and Wear (GB)

(72) Inventors: Colin Douglas Archibald BROWN, Newcastle upon Tyne, Tyne and Wear (GB); Francesca PISAPIA, Newcastle upon Tyne, Tyne and Wear (GB); Donovan Benjamin O'BRIEN, Newcastle upon Tyne, Tyne and Wear (GB)

(21) Appl. No.: 18/685,598

(22) PCT Filed: Aug. 22, 2022
(86) PCT No.: PCT/GB2022/052166

§ 371 Date: Feb. 22, 2024

(30) Foreign Application Priority Data
Aug. 26, 2021 (GB) 2112237.9
Mar. 28, 2022 (GB) 2204371.5

(51) Int. Cl. C12M 1/12; C12M 1/00; C12M 1/42

(52) U.S. Cl. CPC C12M 25/04; C12M 23/04; C12M 29/06; C12M 35/04

(57) ABSTRACT

A fluid flow plate comprising: a fluid reservoir comprising a cavity space defined by a base wall, one or more side walls and an upper wall: wherein the upper wall comprises a plurality of apertures, each adapted to receive a removeable cell culture surface; and wherein, the fluid flow plate is configured to be rocked such that bidirectional flow of fluid in the fluid reservoir is affected. Also described is a kit comprising a fluid flow plate and a method of simulating fluid flow on cells utilising a fluid flow plate.

### TECHNICAL FIELD

The present invention relates to fluid flow plates for simulating fluid flow on cell cultures. In particular, fluid flow plates which are configured to evenly distribute shear stress over a cell culture surface and configured for use with a rocking platform. The present invention also relates to kits and methods for simulating fluid flow and cell cultures.

### BACKGROUND

In vivo, many cell types are exposed to extracellular fluid flow. When culturing cells in an in vitro environment, it is often desirable to recreate as many of the in vivo environmental factors as possible in order to create a more physiologically relevant cell culture. Many cell types that are typically exposed to an extracellular fluid flow in vivo exhibit a physiological response to fluid dynamic stress which is exerted on the cells by the extracellular fluid flow. Therefore, when culturing cells it is desirable to simulate a fluid flow response by exposing the cells to fluid flow in vitro.

There currently exist means with which to expose cells to fluid flow in vitro. For example, microfluidic devices can be used to expose relatively small numbers of cells to small volumes of fluid. One such example of a microfluidic system for this purpose is the Emulate Kidney-Chip model. The Emulate® model utilises a filter support with microfluidic flow over the top surface of the cells. Use of this model with Human Proximal Tubular Epithelial cells has demonstrated that when the cells are exposed to flow, they are more representative of the physiological condition of cells seen in vivo when compared to cells grown without flow.

Another microfluidic model for simulating fluid flow on cells is the Nortis model. The Nortis model cultures cells on the internal surfaces of microfluidic tubes before pumping fluid through the lumen of the tubes. Microfluidic models such as the Emulate® and Nortis models described above are costly to manufacture due to the complexity of the components and requirement to have a means of pumping the fluid through the devices. As a result of their size and cost, it is difficult and costly to conduct high throughput experiments using the existing microfluidic models.

The present invention aims to mitigate the problems associated with the prior art by providing devices, kits and methods of simulating fluid flow on cells cultured in vitro.

### SUMMARY OF THE INVENTION

According to a first aspect of the present invention, there is provided a fluid flow plate comprising:
a fluid reservoir comprising a cavity space defined by a base wall, one or more side walls and an upper wall;
wherein the upper wall comprises at least one aperture, said at least one aperture adapted to receive a removeable cell culture surface;
and wherein the reservoir has a length greater than that of the at least one aperture;
and wherein, the fluid flow plate is configured to be rocked such that bidirectional flow of fluid in the fluid reservoir is effected.

Advantageously, cells on the removable cell culture surfaces can be exposed to flow in the fluid reservoir thereby simulating fluid flow on the cells.

Advantageously, the fluid flow plate is configured to exert 0.2 dyne/cm² to 2 dyne/cm² of shear stress on the removable cell culture surface.

Advantageously, this replicates the shear stress exerted on cells such as proximal tubule cells in vivo and thereby eliciting physiologically relevant response to the fluid flow. Notably, the fluid flow plate can be configured to exert greater than 2 dyne/cm² in cases where a greater shear stress is of interest, for example to provide a disease model to mimic certain disease states.

Preferably the upper wall is sized to receive and retain a conventional cell culture insert, said conventional cell culture insert providing the removable cell culture surface.

Advantageously, conventional cell culture inserts are a readily available and cost-effective means of holding cells in place in the fluid flow plate. Examples of conventional cell culture inserts include Transwell® and ThinCert™ inserts.

Preferably fluid flow plate comprises a means for preventing the rotation of the removable cell culture surface.

Advantageously, the means for preventing the rotation of the removable cell culture surface prevent the removable cell culture surface, such as a cell culture insert, from rotating within the aperture when in use. Unwanted rotation of the removable cell culture surface may adversely affect the fluid dynamics acting on the cells.

The fluid flow plate may for example comprise a male or female portion which can mate with a corresponding female or male portion located on the removable cell culture surface.

Preferably, the means for preventing the rotation of the removable cell culture surface comprises a groove for receiving a protrusion located on the removable cell culture insert. It would be understood that the removable cell culture surface could alternatively comprise a protrusion which is receivable by a groove located on the removable cell culture insert.

Advantageously, a groove on the fluid flow plate configured to receive a protrusion on the removable cell culture surface would be effective in preventing the rotation of the removable cell culture surface in the aperture. Furthermore, many conventional cell culture inserts are provided with a plurality of protrusions and therefore the provision of grooves corresponding to those protrusions would provide a convenient means for preventing their rotation.

Preferably, the rocking motion is provided by a conventional rocker.

Conventional rockers are well known in the art and typically comprise a base electric motor unit which drives a moving platform, deck or other support on which multi-well plates or similar can be placed. The motor speed may be adjustable, to offer a gentle mixing motion through to a more vigorous high-speed mixing action. The platform, deck or support typically has a non-slip surface. Typically, the rocker provides a basic see-saw motion or side-to side rocking motion.

Advantageously, a rocker provides a convenient means for effecting fluid flow in the fluid reservoir.

Optionally the fluid flow plate comprises a plurality of fluid reservoirs.

Advantageously, a fluid flow plate having multiple reservoirs allows for multiple experimental conditions per plate. For example, the fluid in each reservoir may contain different components.

Optionally, each fluid reservoir is in fluid communication with a single aperture.

As such, in use the liquid in the fluid reservoir contacts a single cell culture insert that has been inserted into the aperture.

This allows each cell culture insert to be provided with its own conditions and ensures that any factors released from the cells on a cell culture insert are not transferred to cells on other cell culture inserts and vice versa.

Alternatively, each fluid reservoir is in fluid communication with a plurality of apertures.

This alternative allows any factors released from the cells on a cell culture insert to be in fluid communication with cells on other cell culture inserts via the fluid reservoir.

As such, in use the liquid in the fluid reservoir contacts a plurality cell culture inserts that have been inserted into the plurality of apertures.

Alternatively, the fluid flow plate comprises a plurality of fluid reservoirs at least one of which is in fluid communication with a single aperture and at least one of which is in fluid communication with a plurality of apertures.

Preferably the fluid reservoir is elongate. This would generally be understood to be longer in length than width (or width than length).

Advantageously, the fluid reservoir being elongate, and having a length greater than the length of at least one aperture, provides sufficient space for the fluid to flow in the reservoir. It is preferable that the fluid reservoir extends beyond the length of an aperture on either side of said aperture.

Preferably, the reservoir has a smooth internal profile. Most preferably the reservoir is a rectangular prism shape.

Preferably the height to width ratio of the fluid reservoir is between 1:2 and 1:10, more preferably 1:4 to 1:5. Where the height is measured from the lower wall of the reservoir to the upper wall of the reservoir.

Preferably the fluid flow plate comprises a plate substrate and the reservoir is formed by a recess in the plate substrate.

Optionally, the upper wall of the fluid reservoir comprises a plurality of apertures for receiving the removable cell culture surface, the one or more apertures being arranged substantially opposite the base wall.

Optionally, the upper wall has a plate-like planar surface comprising one or more apertures for receiving the removable cell culture surface and is arranged substantially opposite the base wall.

Preferably each aperture is a channel, said channel being open into the fluid reservoir at a first end and open to the fluid flow plate surface at a second end. The channel extends through the plate-like planar surface.

Optionally the channel is formed by a recess in the plate substrate.

Preferably the fluid flow plate is configured such that a conventional cell culture insert is insertable into the channel, such that in use, a bottom surface of the conventional cell culture insert forms part of the upper wall of the fluid reservoir.

Preferably the bottom surface of the conventional cell culture insert (i.e. the surface that when the insert is inserted faces into the fluid reservoir) is typically a cell growth surface.

Preferably the fluid flow plate is configured such that the removable cell culture surface is arranged to be substantially flush with the surface of the upper wall of the fluid reservoir.

Advantageously, the removable cell culture surface sits substantially flush with said inner surface of the upper wall to provide a continuous inner surface of the upper wall. The provision of a continuous upper wall assists in the smooth flow of the fluid in the fluid reservoir.

Optionally at least a portion of the removable cell culture surface seals the apertures in the upper surface of the fluid reservoir.

Optionally the top of the removable cell culture surface seals the apertures in the upper surface of the fluid reservoir.

Optionally, at least the surfaces proximate to the cell growth surface seal the apertures in the upper surface of the fluid reservoir.

Preferably, the seal is a liquid tight seal.

Advantageously, the liquid tight sealing of the aperture by the removeable cell culture surface prevents liquid, such as cell culture medium, moving from the fluid reservoir into and around the removeable cell culture surface when in use. The liquid tight sealing of the fluid reservoir by the removable cell culture surface allows the liquid in the fluid reservoir to generate shear stress to which cells that are cultured on the cell growth surface can be exposed. Without the liquid tight sealing of the upper wall of the fluid reservoir, liquid in the fluid reservoir is likely to move from the fluid reservoir into and around the removable cell culture surface. This leakage of liquid will affect the fluid dynamics of liquid in the fluid reservoir and subsequently the generation of shear stress.

Preferably, the removeable cell culture surface fits into the aperture by push fit.

Advantageously, the push fit configuration between the removable cell culture surface and aperture provides a liquid tight seal.

Preferably the liquid tight seal is substantially flush with, or substantially on the same plane as, the upper wall of the fluid reservoir.

Advantageously, the liquid tight seal being provided flush with, or substantially on the same plane as, the upper wall of the fluid reservoir provides a continuous upper wall which assists in the smooth flow of fluid in the fluid reservoir.

Preferably the bottom surface of the fluid flow plate is substantially planar.

Advantageously, a planar bottom surface assists in stabilising the fluid flow plate on a flat surface, such as a rocker.

Preferably the fluid flow plate comprises gripping members on the bottom surface.

Advantageously, gripping portions assist in gripping the bottom of the fluid flow plate to a surface, such as a rocker and reduce unintentional movement on a rocker.

Preferably the fluid reservoir comprises at least one fluid inlet.

Advantageously, a fluid inlet allows fluid to be added and/or removed from the fluid reservoir after the removable cell surface has been fitted. Preferably the fluid inlet is separate from the apertures for receiving the removable cell culture insert.

Advantageously, the fluid inlet also provides additional volume into which any liquid in the reservoir, e.g. media, can move when the plate is being rocked from side to side. This allows for efficient mass transfer of the liquid in the reservoir.

Preferably the at least one fluid inlet extends through the upper wall of the fluid flow plate and comprises a first opening, a second opening and at least one side wall; wherein the first opening is open to the fluid reservoir and the second opening is open to the surface of the upper wall of fluid flow plate, said second opening being arranged substantially opposite said first opening; and wherein said first opening is connected to said second opening by the at least one side wall.

Preferably the inlet is provided substantially at, at or in close proximity to, an end of the reservoir.

Preferably each reservoir is provided with at least two inlets. Preferably said at least two inlets are provided at either end of the reservoir.

Preferably, an inlet is a different shape to an aperture. Preferably an inlet is shaped such that it will not receive the removable cell culture surface.

In a less preferred embodiment the inlet may not have the second opening to the surface. In this case it cannot be used for the additional or removal of fluid such as liquid media and simply acts as a volume space into which said fluid can move when the fluid flow plate has a rocking motion applied thereto.

Typically, the volume space provided by the inlet is provided in a plane above the plane of the reservoir—this allows vertical movement of liquid from the reservoir within or into the inlet when the plate is rocked.

Preferably, the fluid flow plate is configured to exert substantially uniform shear stress across the removable cell culture surface.

Advantageously, the cells on the removable cell surface are subjected to a substantially uniform shear stress resulting in a substantially uniform physiological response to said shear stress.

According to a second aspect of the present invention, there is provided a kit comprising a fluid flow plate in accordance with the first aspect and a removeable cell culture surface.

Preferably the removable cell culture surface is a conventional cell culture insert.

Optionally the kit further comprises a conventional rocker device.

Preferably the conventional cell culture insert comprises an opening, a base and at least one side wall, said base being arranged substantially opposite said opening.

Preferably the conventional cell culture insert comprises a well, said well formed by the base and at least side wall of the conventional cell culture insert.

Preferably the base of the conventional cell culture insert comprises a first base wall within the well and a second base wall outside the well, wherein said second base wall is arranged to be substantially parallel with said first base wall.

Preferably the removable cell culture surface is configured such that cells can be cultured on at least a part of the second surface of the conventional cell culture insert.

Advantageously, when cells are grown on the external surface of the conventional cell culture insert the cells will be in fluid communication with the fluid reservoir of the fluid flow plate when the cell insert is in place in the fluid flow plate.

According to a third aspect of the present invention, there is provided a method of simulating fluid flow on cells comprising;

fixing a removable cell culture surface to the fluid flow plate of the first aspect of the present invention;

wherein the removable cell culture surface comprises cells;

and exerting a rocking motion on the fluid flow plate;

wherein the fluid flow plate comprises fluid and the rocking motion effects the bidirectional flow of fluid in the fluid flow plate.

Preferably the rocking motion is provided by a rocker device.

Preferably the plate is rocked by the rocker device at a frequency of 0.1 cycles per minute at an angle of 7 degrees. In other embodiments the plate is rocked by the rocker device between 7 and 14 times per minute at an angle between 11 and 17 degrees.

Various further features and aspects of the invention are defined in the claims.

Unless defined otherwise, all technical and scientific terms used herein have the meaning commonly understood by a person skilled in the art to which this invention belongs.

### BRIEF DESCRIPTION OF THE DRAWINGS

Embodiments of the present invention will now be described by way of example only with reference to the accompanying drawings where like parts are provided with corresponding reference numerals and in which:

FIG. 1 shows a series of simplified schematic diagrams of a fluid flow plate according to certain embodiments of the present invention;

FIG. 2 shows a top-down view of a fluid flow plate according to certain aspects of the present invention;

FIG. 3 shows a top-down view of an alternative embodiment of a fluid flow plate according to certain aspects of the present invention;

FIG. 4 shows a series of simplified schematic diagrams of a fluid flow plate in use with a rocker.

FIG. 5A shows a velocity magnitude slice plot of the fluid reservoir. FIG. 5B shows shear stress profile across the width of the fluid flow reservoir in the region configured to receive a removable cell culture surface. FIG. 5C shows a velocity profile across the width of the fluid flow reservoir in the region configured to receive a removable cell culture surface.

### DETAILED DESCRIPTION

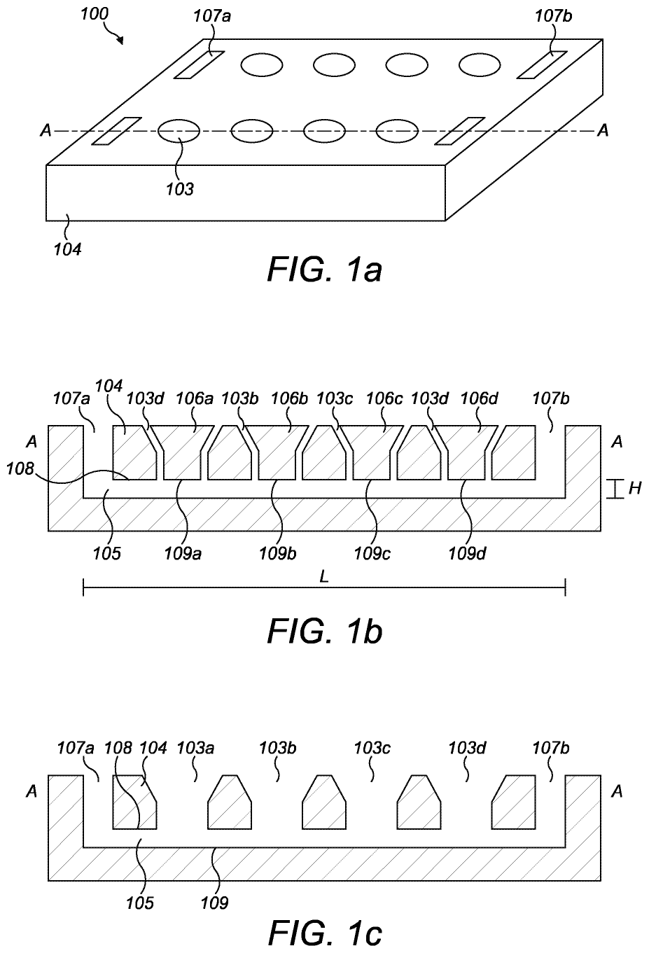

FIG. 1 shows a perspective view and cross sections of a fluid flow plate taken through the apertures in the plate according to certain embodiments of the present invention. FIG. 1A shows a perspective view of a fluid flow plate 100. The fluid flow plate comprises eight apertures 103 and two fluid reservoirs (not shown). It would be clear to a person skilled in the art that fluid flow plates according to the present invention could encompass any number of apertures and fluid reservoirs. For example, a fluid flow plate in accordance with the present invention may comprise 24 apertures and 4 fluid reservoirs. Alternatively, a fluid flow plate in accordance with the present invention may incorporate 48 apertures and 8 fluid reservoirs.

The apertures 103 are arranged in two parallel rows, each row containing four apertures 103. Each row of four apertures also comprises two inlets 107a, 107b which are arranged such that the four apertures are located between the two inlets 107a, 107b. Each row comprises a fluid reservoir (not shown). The four apertures and two inlets of each row lead to the fluid reservoir. The fluid reservoir runs between the two inlets 107a, 107b. The apertures 103 are configured to receive a removable cell culture surface such as a conventional cell culture insert.

It would be understood that each row may comprise only one inlet, or that an aperture may also act as an inlet (although it is generally preferred to have a separate inlet).

The fluid flow plate is manufactured from a plate substrate 104. The plate substrate is typically a plastic such as polystyrene. The fluid flow plate may be manufactured by 3-dimensional (3D) printing. The fluid flow plate may be manufactured as a single integral device. Alternatively, the fluid flow plate may be manufactured as separate components which are subsequently fixed by any suitable means such as bonding or welding. The fluid flow plate may further comprise a cover member to cover the apertures.

The dashed line (A-A) indicates the plane of the cross sections shown in FIGS. 1B and 1C.

FIG. 1B shows a cross section of the plate depicted in FIG. 1A taken through the plane indicated by the dashed line (A-A) and with removable cell culture surfaces in place. The removable cell culture surfaces 106a, 106b, 106c, 106d are conventional cell culture inserts, such as Transwell® inserts.

The cell culture inserts 106a, 106b, 106c, 106d fit into the apertures 103a, 103b, 103c, 103d such that the top surface of the cell culture inserts 106a, 106b, 106c, 106d sit substantially flush with the top surface of the fluid flow plate 100. The bottom of the cell culture inserts 106a, 106b, 106c, 106d sit substantially flush with the upper wall 108 of the fluid reservoir 105.

The fluid reservoir 105 is delimited on the four side walls and base wall by the plate substrate 104. The upper wall of the fluid reservoir 105 is partially delimited by the cell culture surfaces 109a, 109b, 109c, 109d of the cell culture inserts 106a, 106b, 106c, 106d and partially by the plate substrate 104.

The insertion of the cell culture inserts 106a, 106b, 106c, 106d into the apertures 103a, 103b, 103c, 103d provide a liquid tight seal such that liquid cannot move from the fluid reservoir 105 into and/or around the cell culture inserts 106a, 106b, 106c, 106d.

The inlets 107a, 107b are open to the top of the fluid flow plate on a first end and open to the fluid reservoir 105 at a second end. The four side walls of the inlets 107a, 107b are delimited by the plate substrate 104. The inlets 107a, 107b can be used to conveniently add and/or remove fluid from the fluid reservoir or to add components to the fluid reservoir when in use, without the need to remove any of the cell culture inserts 106a, 106b, 106c, 106d to access the fluid reservoir.

The fluid flow plate 100 is arranged such that, when in use, cells grown on the cell culture surfaces 109a, 109b, 109c, 109d of the cell culture inserts 106a, 106b, 106c, 106d are in fluid communication with fluid in the fluid reservoir 105. Fluid can be added and/or removed from the fluid reservoir 105 via the inlets 107a, 107b. The fluid flow plate 100 is configured to be used with a conventional rocker. The angle and speed of the rocker on which the fluid flow plate 100 sits determine the fluid flow rate of the fluid in the fluid reservoir. The fluid flow plate 100 is configured such that the shear stress applied to the cells on the cell culture surface 109a, 109b, 109c, 109d is substantially equal across the entire surface of the cell culture surfaces 109a, 109b, 109c, 109d.

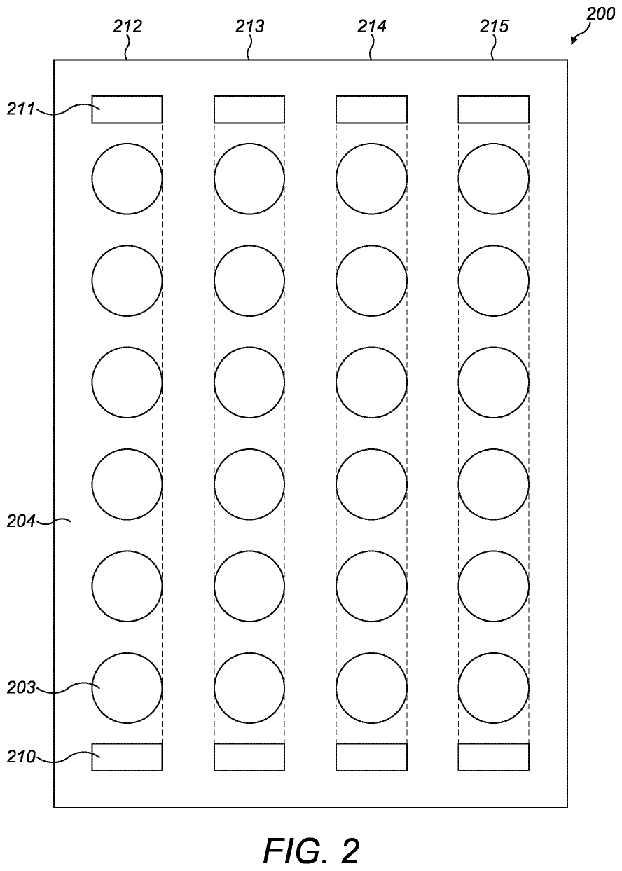

The apertures 103a, 103b, 103c, 103d are configured to have a diameter substantially the same as the width of the fluid flow reservoir 105 (shown more clearly in FIG. 2). This configuration allows fluid flow across the entire cell culture surfaces 109a, 109b, 109c, 109d.

Preferably, the apertures are substantially the same size as the wells of a conventional cell culture plate. The height of the fluid reservoir is the distance between the base wall 109 and upper wall 108 (H). The width of the fluid reservoir is the distance between the two side walls (not shown) and is configured to be substantially the same as the diameter of the apertures 103. For example, the fluid reservoir may have a height of 3 mm and a width of 9 mm. The length of the fluid reservoir is indicated on FIG. 1B (L) but is greater than 9 mm.

FIG. 1C shows a cross section of the fluid flow plate of FIG. 1A taken across the plane indicated by the dashed line (A-A) without cell culture inserts fitted. The apertures 103a, 103b, 103c, 103d are formed by recesses in the plate substrate 104. When there are no cell culture inserts in place, the upper wall 108 of the fluid reservoir 105 is not continuous.

FIG. 2 shows a fluid flow plate according to certain aspects of the present invention. The fluid flow plate 200 comprises four rows 212, 213, 214, 215. Each row comprises a first fluid inlet 210 at a first end of the plate substrate 204 and a second fluid inlet 211 at a second end of the plate substrate 204. Each fluid inlet 210, 211 is open at a first end to the plate substrate surface and at a second end to a fluid reservoir. The placement of the fluid reservoirs is depicted by dashed lines.

On each row 212, 213, 214, 215, placed between the first and second fluid inlet, are six apertures 203. Each aperture is open to the plate substrate surface at a first end and to the fluid reservoir at a second end, said second end being located substantially opposite said first end. Between the first end and second end of the aperture 203 is a wall which extends around the circumference of the aperture 203, connecting the first end of the aperture 203 to the second end of the aperture 203. The wall of the aperture is delimited by the plate substrate 204. The apertures are configured to receive a conventional cell culture insert.

Each of the four rows 212, 213, 214, 215 is associated with a distinct fluid reservoir, into which each of the six apertures located on each row is open. Each reservoir is a cavity which extends between the first and second fluid inlets 210, 211, such that the first and second fluid inlets 210, 211 are in fluid communication via the fluid reservoir.

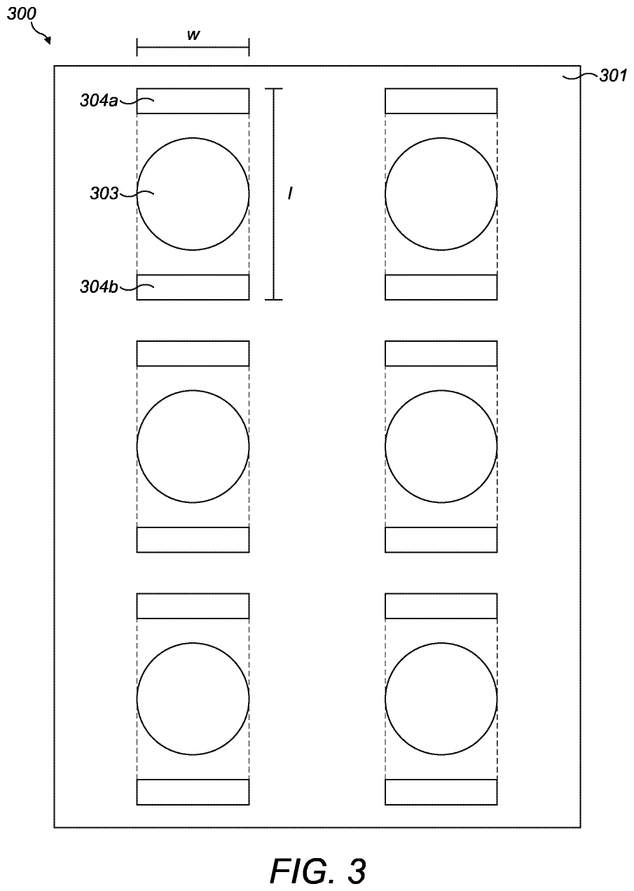

FIG. 3 shows an alternative embodiment of the fluid flow plate in accordance with certain embodiments of the present invention. The fluid flow plate 300 comprises six apertures 303 suitable for receiving a removable cell culture surface. It would be obvious to the skilled person that a fluid flow plate could be provided with any number of apertures. For example, a fluid flow plate may contain 4 apertures, 24 apertures or 96 apertures. Each aperture 303 is associated with a fluid reservoir (dashed lines). The fluid reservoir is a cavity into which the aperture 303 opens. Advantageously, each aperture 303 being associated with a separate fluid reservoir enables the use of a different experimental condition for each removeable cell culture surface.

Each aperture 303 and fluid reservoir is also associated with two fluid inlets 304a, 304b. The fluid inlets 304a, 304b are channels open to the top of the fluid flow plate at a first end and open to the fluid reservoir at a second end. The two ends are connected by 4 side walls. The 4 side walls are provided by the plate substrate 301. The fluid inlets 304a, 304b are used to add and/or remove fluid from the fluid reservoir. The fluid inlets 304a, 304b also provide an overflow whereby fluid in the fluid reservoir can collect when the fluid flow plate is being rocked. The fluid inlets have a depth of 18-20 mm and a length (/) of 4 mm. The depth of the fluid inlet is the distance from the opening on the surface of the fluid flow plate to the fluid reservoir. The depth to length ratio of the fluid inlet is between 5:1 and 4:1.

The fluid reservoir associated with each aperture is elongate. The fluid reservoir has a width (w) which is substantially the same as the width of the aperture. The length (/) of the fluid reservoir, i.e. the distance between the two inlets, is larger than the width (w). The fluid reservoir being elongate is important to ensure sufficient fluid flow in the fluid reservoir. It is also preferred, as shown in this embodiment that the reservoir has fluid inlets 304a, 304b at either end of the reservoir.

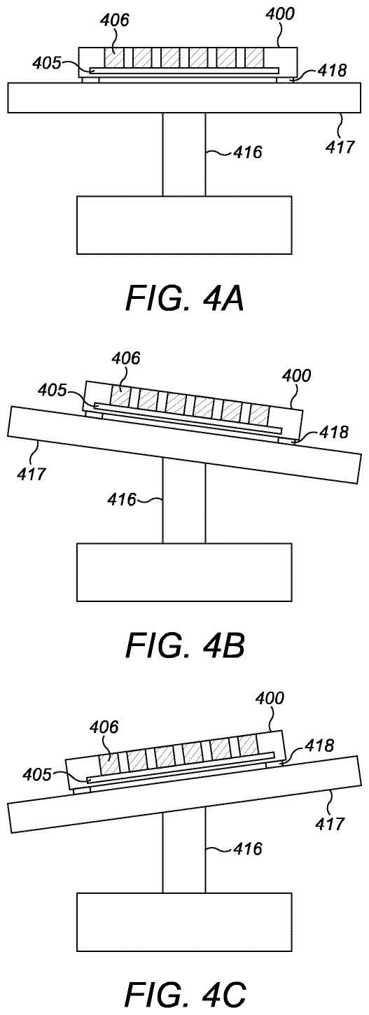

FIG. 4 shows a series of diagrams depicting the fluid flow plate in use with a conventional rocker.

The fluid flow plate 400 comprises a fluid reservoir 405 into which fluid, such as cell culture media can be added. The fluid flow plate 400 comprises a plurality of apertures which are configured to each receive a removable cell culture surface such as a conventional cell culture insert 406. The fluid flow plate is configured such that, when in use, the fluid in the fluid reservoir 405 is in fluid communication with cells cultured on the cell culture insert 406.

In order to effect fluid flow in the fluid reservoir, the fluid flow plate 400 is placed on a rocker 416. In FIG. 4A, the fluid flow plate 400 is placed on the platform 417 of the rocker 416. The rocker 416 and fluid flow plate 400 are in a substantially level configuration. A substantially level configuration is considered to be approximately 0°. The rocker 416 then tilts the platform 417 from side to side to effect fluid flow in the fluid reservoir 405. The degree of tilt of the rocker platform 417 and frequency at which the platform 417 is tilted determines the fluid flow rate of the fluid in the fluid reservoir 405. This will in turn determine the shear stress which any cells with the reservoir are exposed to. For example, when working with proximal tubule cells, normal physiological conditions result in an exerted shear stress on the cells of 0.2 to 2 dyne/cm². Having the rocker 416 configured to tilt the platform 417 at an angle of ±7° at 0.1 cycles per minute can replicate such conditions. In other embodiments the rocker 416 may be configured to tilt the platform 417 10 times per minute at an angle of ±14°. FIGS. 4B and 4C depict the fluid flow plate 400 on a platform tilted in a first and second direction.

It will be understood that the tilt angle and frequency of tilt cycles (i.e. rocking speed) can be easily adjusted. The algorithms for variable speed settings and tilt angles are well known in the art and integrated into conventional rockers.

The fluid flow plate 400 may comprise one or more gripping member 418. The gripping member assists in increasing the friction between the fluid flow plate 400 and platform 417 to reduce any unintended movement of the fluid flow plate 400 on the platform 417 when in use. The gripping member may be a plurality of small gripping feet 418 attached to the bottom surface of the fluid flow plate 400. The small gripping feet 418 comprise a high friction material such as rubber, which increases the friction between the bottom surface of the fluid flow plate 400 and the surface of the platform 417.

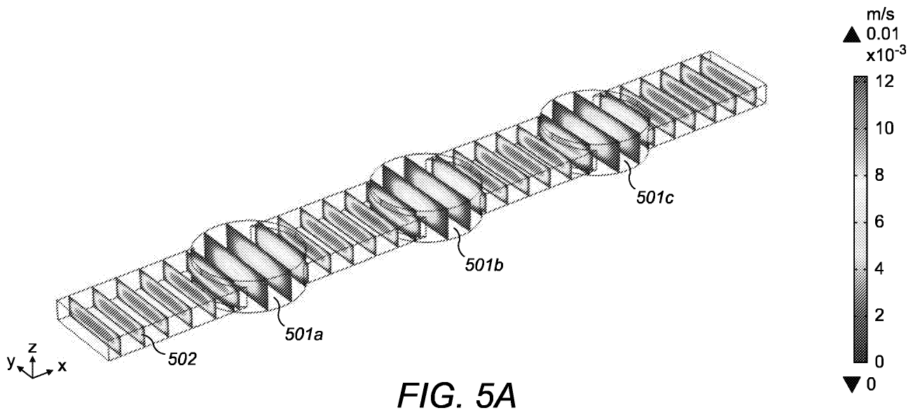

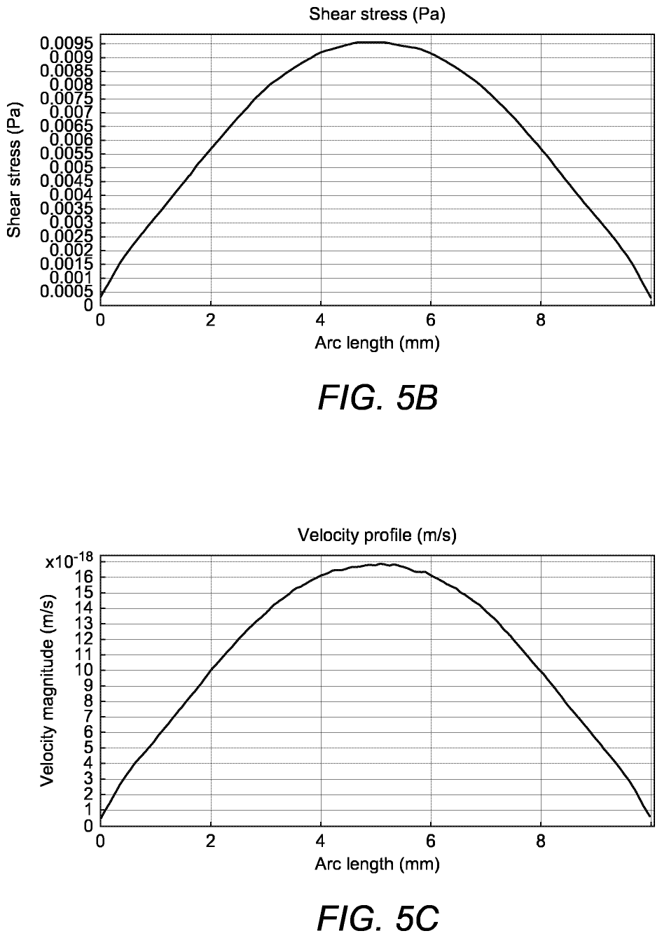

FIG. 5A shows a velocity magnitude slice plot of the fluid reservoir. FIG. 5A demonstrates the velocity magnitude across the fluid flow reservoir 502 for a shear stress of 0.2 dyne/cm² (0.02 Pa). The fluid reservoir 502 comprises three apertures for receiving a removable cell culture surface 501a, 502b, 501c. The velocity magnitude, and therefore shear stress is substantially equal across the surface of the removable cell culture surface 501a, 502b, 501c.

FIG. 5B shows shear stress profile across the width of the fluid flow reservoir in the region configured to receive a removable cell culture surface.

FIG. 5C shows a velocity profile across the width of the fluid flow reservoir in the region configured to receive a removable cell culture surface.

### Experimental Data

All of the features disclosed in this specification (including any accompanying claims, abstract and drawings), and/or all of the steps of any method or process so disclosed, may be combined in any combination, except combinations where at least some of such features and/or steps are mutually exclusive. Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features. The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims are generally intended as “open” terms (e.g., the term “including” or “comprising” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

It will be appreciated that various embodiments of the present disclosure have been described herein for purposes of illustration, and that various modifications may be made without departing from the scope of the present disclosure. Accordingly, the various embodiments disclosed herein are not intended to be limiting, with the true scope being indicated by the following claims.

1. A fluid flow plate comprising:
a fluid reservoir comprising a cavity space defined by a base wall, one or more side walls and an upper wall having a plate-like planar surface;
wherein the upper wall comprises at least one aperture adapted to receive a removeable cell culture surface, said aperture being a channel which is open into the fluid reservoir at a first end and open to the fluid flow plate surface at a second end;
wherein the reservoir has a length greater than that of the at least one aperture; characterised in that the fluid flow plate is configured such that when a removeable cell culture surface is inserted into the channel, a bottom surface of the removeable cell culture surface forms part of the upper wall of the fluid reservoir and the removable cell culture surface is arranged to be substantially flush with the surface of the upper wall of the fluid reservoir
and, the fluid flow plate is configured to be rocked such that bidirectional flow of fluid in the fluid reservoir is effected.

2. The fluid flow plate of claim 1, wherein the upper wall is sized to receive and retain a conventional cell culture insert, said conventional cell culture insert providing the removable cell culture surface.

3. The fluid flow plate of claim 1, wherein the fluid reservoir comprises at least one fluid inlet.

4. The fluid flow plate of claim 3, wherein the at least one fluid inlet comprises a first opening, a second opening and at least one side wall;
wherein the first opening is open into the fluid reservoir and the second opening is open to the surface of the fluid flow plate, said second opening being arranged substantially opposite the first opening;
and wherein said first opening is connected to said second opening by the at least one side wall.

5. The fluid flow plate of claim 1, comprising a means for preventing the rotation of the removable cell culture surface.

6. A fluid flow plate according to claim 5, wherein the means for preventing the rotation of the removable cell culture surface comprises a groove for receiving a protrusion located on the removable cell culture surface.

7. The fluid flow plate of claim 1, comprising a plurality of fluid reservoirs.

8. The fluid flow plate of claim 7 wherein each fluid reservoir is in fluid communication with a single aperture.

9. The fluid flow plate of claim 1 wherein the fluid reservoir is in fluid communication with a plurality of apertures.

10. The fluid flow plate of claim 1, wherein the fluid flow plate comprises a plate substrate.

11. The fluid flow plate of claim 10, wherein the fluid reservoir is formed by a recess in the plate substrate.

12. The fluid flow plate of claim 11, wherein the channel is formed by the recess in the plate substrate.

13. The fluid flow plate of claim 1, wherein the bottom surface of the fluid flow plate is substantially planar.

14. The fluid flow plate of claim 1, comprising gripping members on the bottom surface.

15. The fluid flow plate of claim 1, configured to exert substantially uniform shear stress across the removable cell culture surface.

16. A kit comprising the fluid flow plate of claim 1, and a removeable cell culture surface.

17. The kit of claim 16, further comprising a conventional rocker device.

18. The kit of claim 16, wherein the removeable cell culture surface is a conventional cell culture insert and comprises an opening, a base and at least one side wall, said base being arranged substantially opposite said opening.

19. The kit of claim 18, wherein the conventional cell culture insert comprises a well, said well formed by the base and at least side wall of the conventional cell culture insert.

20. The kit of claim 19, wherein the base of the conventional cell culture insert comprises a first base wall surface within the well and a second base wall surface outside the well, wherein said second base wall surface is arranged to be substantially parallel with said first base wall surface and cells can be cultured on at least a part of the second base wall surface of the conventional cell culture insert.

21. A method of simulating fluid flow on cells comprising;
fixing a removable cell culture surface to a fluid flow plate of claims 1-86-44, wherein the removable cell culture surface comprises cells;
and exerting a rocking motion on the fluid flow plate;
wherein the fluid flow plate comprises fluid and the rocking motion effects the bidirectional flow of fluid in the fluid flow plate.

22. The method of claim 21, wherein the rocking motion is provided by a rocker device arranged to exert a rocking motion with a specifiable angle and cycle frequency.
